(19) United States
(12) Patent Application Publication
(10) Pub. No.: US 2019/0247607 A1
(43) Pub. Date: Aug. 15, 2019

(54) INHALATOR

(71) Applicant: LK Investment A/S, Humlebæk (DK)

(72) Inventor: Carsten Leonhard Knudsen, Neksø (DK)

(21) Appl. No.: 16/243,282

(22) Filed: Jan. 9, 2019

Related U.S. Application Data
(63) Continuation of application No. 14/423,080, filed on Feb. 20, 2015, now abandoned.
(60) Provisional application No. 61/700,805, filed on Sep. 13, 2012.

(30) Foreign Application Priority Data
Aug. 20, 2012 (DK) PA 2012 70489

(51) Int. Cl. A61M 16/14; A61M 16/20; A61M 15/00; A24F 47/00; A61M 15/06

(52) U.S. Cl. CPC A61M 16/14; A61M 16/20; A61M 15/0091; A61M 15/002; A24F 47/002; A61M 15/0003; A61M 15/06; A61M 15/0021

(57) ABSTRACT

An inhalator (1) comprising
a first compartment (21) containing an active compound;
a second chamber (31) containing a transporter and
an activation part (40) configured for regulating an air flow through the inhalator (1), and arranged at a distal end the second compartment (31),
characterized in that the first compartment (21) is arranged between a user interface part (10) and the second compartment (31) such that when a user sucks air through the inhalator (1), the transporter is drawn from the second compartment (31) through the first compartment (21), whereby the active compound is captured by the transporter and flows into the user interface part (10) and the user.

The present invention relates to an improved inhalator, also called an inhaler. The inhalator may allow two or more substances to be packed such that they do not interact until the inhalator is brought to an operative state.

### BACKGROUND OF THE INVENTION

Often there is a need to store two or more reactive substances together but separated, e.g. two component glue, penicillin and water or interactive substances. When mixed, such substances will often initiate a reaction.

It is known that reactive substances when mixed can degrade each other by oxidation, reduction or by forming combination products. Likewise, the presence of air can sometimes interact with substances in an unwanted way. In connection with inhalators where two or more substances need to be mixed and inhaled it is often necessary to store the substances separately prior to inhalation.

EP 2002856 discloses an inhalator comprising a number of elastic bodies or spheres elastically restrained in a duct. Prior to use, the substance or substances, which are to be released and inhaled by the user, are kept in the chambers defined by the space between two or more spheres. The inhalator can be activated by inserting a pin into the duct container, and the pin will push the elastic spheres into the end piece. Substances previously kept in different chambers are thus allowed to mix and blend and are exposed to air whereby the different drugs or substances evaporate and are made available for the user to inhale. The pin is then removed whereby a free passage of air is provided, and the user can inhale the substances through the mouth piece. The spheres are made of for instance compressible silicone, rubber, neoprene or the like. In some applications, the use of a special skin forming, cell foamed silicone can be advantageous.

The above inhalator has been shown to give rise to i.e. the following problems.
the pin must be discarded;
a high air flow through the inhalator leads to an inefficient use of the stored substances;
liquid present in the inhalator may flow out of the inhalator through the end opposite the mouth piece;
inefficient and incomplete spending of some of the substances.

### DISCLOSURE OF THE INVENTION

It is an object of the present invention to provide an improved inhalator i.e. solving the above problems.

These objects are achieved by an inhalator according to claim 1. The inhalator may further comprise the features of any one of the claims 1-5, or any combination thereof.

In another aspect, the objects are achieved by an inhalator according to claim 2;

In another aspect, the objects are achieved by an inhalator according to claim 3;

In yet another aspect, the objects are achieved by an inhalator according to claim 4;

A leak free inhalator is achieved by the aspects of the invention according to claim 5.

In yet another aspect, the objects are achieved by an inhalator according to claim 6;

Other alternative embodiment of the invention is disclosed in detailed description below.

If the inhalator may be used as a smoke-free cigarette, i.e. for inhaling nicotine, it is advantageous to store the nicotine substance without exposure the air/oxygen. If contact with ambient air can be avoided, the durability of the product will be significantly longer. Further, if any aromatic substance or substances or helping agents are to be inhaled in combination with the nicotine, these aromatic substances are advantageously kept in one or more chambers separate from the nicotine substance, in order to avoid reaction between the substances.

The aromatic substances could be of the group of Beta damascenone, 4-oxo-beta-ionone, oxo-edulan I-II, 3-oxo-alpha-ionone, Dihydroactionodiolide, Megastigmatrienones (4 isomer), different Carotinoide derivate The helping agents could for instance be different air humidifying agents, pH regulating fluids (such as picrine acid or ammonia), propylene glycol, catalysts, emulsifiers, Tannin (astringent) or different naturopathic drugs.

The different parts and modules of the inhalator can be made of different plastic materials, metal alloys. Barex metal alloys, or silicone to give the user a more realistic feeling of holding and ‘smoking’ a cigarette, the inhalator is in one embodiment wrapped with cigarette and filter paper.

The inhalator may also be used for dispensing other tobacco substances, e.g. as an alternative to chewing tobacco.

The inhalator may also be used for dispensing other substances, flavors (smells and/or tastes).

The inhalator may also be used to orally dispense medicines or to dispense medicines via a person's nose.

### BRIEF DESCRIPTION OF THE DRAWINGS

In the following detailed portion of the present description, the invention will be explained in more detail with reference to the exemplary embodiments shown in the drawings, in which:

FIG. 1 is a perspective view of an inhalator,

FIG. 2, in a sectional side view, shows an inhalator according to an aspect of the invention:

FIG. 3A, show details of a valve of the inhalator shown in FIG. 2;

FIG. 3B, in an exploded perspective view, shows details of the valve shown in FIG. 3A, when the valve is closed;

FIG. 3C, in an exploded perspective view, shows details of the valve shown in FIG. 3A, when the valve is open;

FIG. 30 in a sectional side view, shows details of a rotation mechanism of parts of the inhalator,

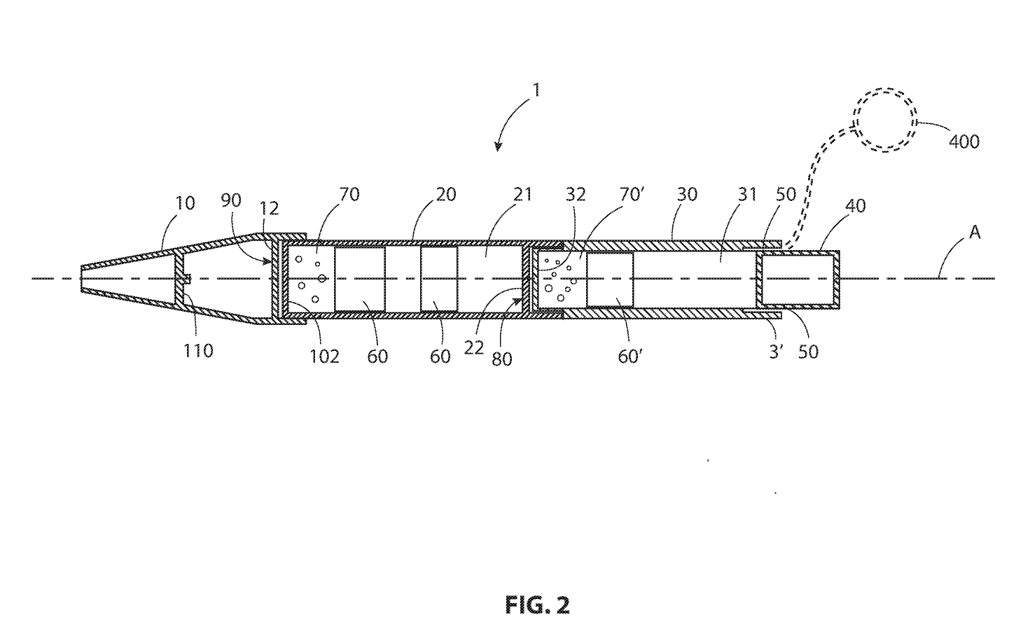

FIG. 4, in a sectional side view, shows details of one embodiment of a valve between a user interface part and a first tubular part of the inhalator shown in FIG. 2;

FIG. 5A, in a side sectional view, shows a user interface part according to an embodiment of the invention, and with a lattice;

FIG. 58 shows a section taken along B-B in FIG. 5A;

FIG. 5C shows a section taken along C-C in FIG. 5A;

FIG. 50 shows the interface part form FIG. 5A in a rear view, i.e. from the distal side, or the side facing a first tubular part:

FIG. 5E, in a perspective view, shows a lattice as in FIGS. 5A-D and as formed e.g. by the tool shown in FIG. 6;

FIG. 6 shows a molding tool for injection molding a lattice as shown in FIGS. 5A-E in a user interface part;

FIG. 7A, in a sectional side view, shows details of one embodiment of a valve between a second tubular part and an activation part of the inhalator shown in FIG. 2;

FIG. 78, in a partly see-through perspective view, shows an activation part, according to one embodiment;

FIGS. 8A-C show an inhalator according to another aspect of the invention;

FIGS. 9A-G show a mouthpiece for an inhalator, where the mouthpiece is shown in a perspective view in FIG. 9A, in a side sectional view in FIG. 9B, in a top sectional view in FIG. 9C and in a rear view in FIG. 9C, and where details of a filter shown in the circle in FIG. 9C is shown in FIG. 9E. FIG. 9F shows the mouthpiece in a perspective views from the rear, and FIG. 9C show a partly cut, perspective view of the mouthpiece; and

FIG. 10 shows an inhalator which may be particularly suitable for delivering a medical compound.

### DETAILED DESCRIPTION OF SELECTED EMBODIMENTS

In the following detailed description of an inhalator according to the invention will be described by the preferred embodiments.

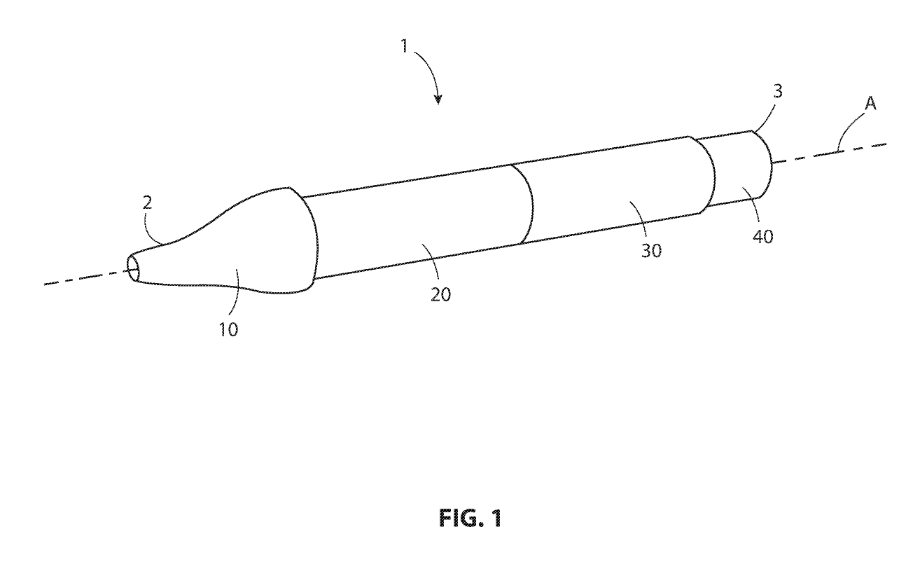

FIG. 1 shows in an inhalator 1 comprising an elongated tubular, e.g. cylindrical, housing. The elongate tubular housing has a proximal end 2 and a distal end 3, and a longitudinal axis A. By proximal end is meant the end that is closest to a user of the inhalator during use and by distal end is meant the end that faces away from the user during use. At the proximal end 2, the inhalator 1 may as shown comprise a user interface part 10 configured for allowing the user to inhale one or more substances enclosed in the inhalator either through a mouth or a nose of a user. Thus the user interface part 10 may be a mouthpiece or a part specially adapted for interaction with nostrils of the nose of a user.

The inhalator 1 further comprises a first and a second tubular part 20, 30 arranged in extension of each other. The first tubular part 20 defines a first compartment 21 and the second tubular part defines a second compartment 31. The first tubular part 20 is arranged between the user interface part 10 and the second tubular part. Thus the first compartment 21 is arranged between the user interface part 10 and the second compartment 31.

At the distal end 3 of the inhalator 1 an activator part 40 is arranged in extension of the second tubular part 30, as seen along the longitudinal axis A of the inhalator. The activator part 40 controls one or more venting ports 50 arranged at a distal end 3′ of the second tubular part. The one or more venting ports 50 allows ambient air to pass through the inhalator 1 from the venting ports 50 through the second compartment 31, the first compartment 21 and the user interface part 10, e.g. by the user providing a suction at the proximal end 2 of the inhalator 1. Alternatively, a source 400 of flowing/pressurized gas, e.g. ambient air, may be attached or located in connection to the venting ports 50 to provide a flow through the inhalator from the proximally arranged inhalation ports 50 to the user interface part 10 at the proximal end 2 of the inhalator 1. Such a source of flowing gas may in one embodiment, and as indicated in FIG. 2 be provided by a valved resilient ball connected to the venting port through suitable tubing. Other sources of pressurized gas may be used. By providing a gas flow through the inhalator molecules or particles or flakes of a compound contained in the first and/or second compartments 21, 31 may be moved from the first and/or second compartments 21, 31 through the user interface part 10 and into the mouth or nose of the user.

The activator part 40 is configured for opening and closing the venting ports 50. The activator part 40 may further be configured for adjusting the volume of ambient air that may pass through the inhalator 1 by adjusting the size, e.g. ea cross-sectional area, of the venting port 50.

A typical size of the inhalator 1 could be similar to that of a standard cigarette. However, dependent on the use and the type and volume/amount/dosage of substance to be delivered from the inhalator 1, the size of the inhalator may be chosen differently.

FIG. 2 shows a side sectional view of an inhalator according to an embodiment of the invention, and the inhalator shown in FIG. 1.

The first compartment 21 contains an active compound to be delivered to the user. The active compound may be a form of medicine, herbs, tobacco, a tobacco extract including nicotine, pure nicotine. Preferably, the active compound is contained in one or more diffusion open material plugs 60 arranged in the first compartment 21. The active compound may have been loaded into the diffusion open material plugs 80 prior to the mounting of the plug 60 in the first compartment 21. The diffusion open material plugs 60 fits, preferably tightly into the first compartment 21. i.e. a cross-sectional area of the plugs 60 is adapted to be approximately the same as the cross-sectional area of the first compartment 21. The cross sectional shape of the plugs 60 is configured to correspond the cross sectional shape of the first compartment 21. Preferably, the cross-sectional shape of the first compartment 21 and the plugs 60 is circular, i.e. the plugs 60 are straight cylindrical. The plugs 60 are made in a diffusion open material to allow a flow or gas and/or liquid or at least liquid drops or droplets through the plug 60. An example of a suitable material could be polypropylene, PP. The one or more plugs 60 may fill out the first compartment 21 fully or only partially (in a direction along the longitudinal axis A). A filling 70 may further be provided, when the plug 80 or plugs 60 only partially occupies the space defined by the first compartment 21 (in a direction along the longitudinal axis A). Such a filling 70 may be provided by grains of rice, flakes of tobacco leaves and/or other, provided that the filling material packs to allow flow of a gas and/or liquid through the compartment 21.

The second compartment 31 contains a transport compound, the transport compound being of a kind suitable for interacting with the molecules, particles or flakes contained in the first compartment 21 when a gas flows through the inhalator and moves the transport compound from the second compartment 31 through the first compartment 21, to transport the active compound out of the first compartment 21 through the user interface part 10 and into the users mouth or nose.

The transport compound may e.g. be and aromatic compound. The transport compound is a compound in the form of a molecule, a particle, or a flake with a size/weight ratio that allows the individual components of the compound to be transported when influenced by an air flow through the inhalator. The transport compound further must have the ability to interact with the active compound, which typically will have a size/weight ratio that prevents it from being transported by the gas flow through the inhalator 1. By interaction is meant chemical binding, or kinetic or thermal interaction.

Preferably, the transport compound is loaded into one or more diffusion open material plugs 60′ arranged in the second compartment 31. In FIG. 2 only a single plug 60′ is shown. The diffusion open material plugs 60′ fits, preferably tightly into the second compartment 31, in the same manner as the plugs 60 in the first compartment 21. However, the cross-sectional area or the diameter of the two compartments may not be the same. The cross sectional shape of the plugs 60′ is configured to correspond the cross sectional shape of the second compartment 31. Preferably, the cross-sectional shape of the second compartment 31 and the plugs 60′ is circular, i.e. the plugs 60′ are straight cylindrical. The plugs 60′ are made in a diffusion open material to allow a flow or gas and/or liquid or at least liquid drops or droplets through the plug 60′. An example of a suitable material could be polypropylene, PP. The one or more plugs 60′ may fill out the second compartment 31 fully or only partially (in a direction along the longitudinal axis A). A filling 70′ may further be provided, when the plug or plugs 60′ only partially occupies the space defined by the second compartment 31. Such a filling 70′ may be provided by grains of rice, flakes of tobacco leaves or other, provided that the filling material packs to allow flow of a gas and/or liquid through the compartment 31.

In FIG. 2 two venting ports 50 are shown in the shape of ducts formed parallel to the longitudinal axis A. and in an inner surface of the compartment 31 at the distal end 3′ thereof. By pulling the actuation part 40 in the distal direction, i.e. away from the user interface part 10, a portion of the ducts forming the ventilation ports 50 may open into the compartment 31 and thus provide communication with the ambient air (or a source of pressurized gas as described above). Thus FIG. 2 shows the inhalator 1 in an activated state. In an inhalator 1 in an unactivated state, the actuation part 40 would be further toward the proximal end 2 of the inhalator 2 where proximal ends of the ducts forming the ventilation ports 50 would cover be covered by actuation part 40. Other embodiment of one or more ventilation ports 50 are described below.

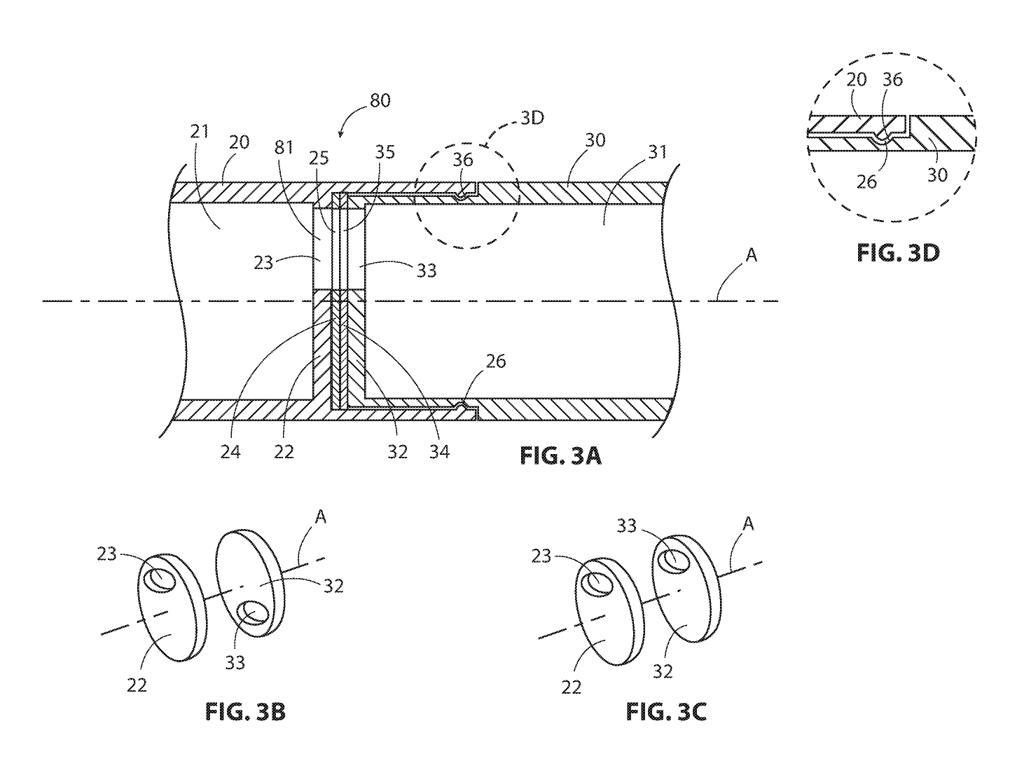

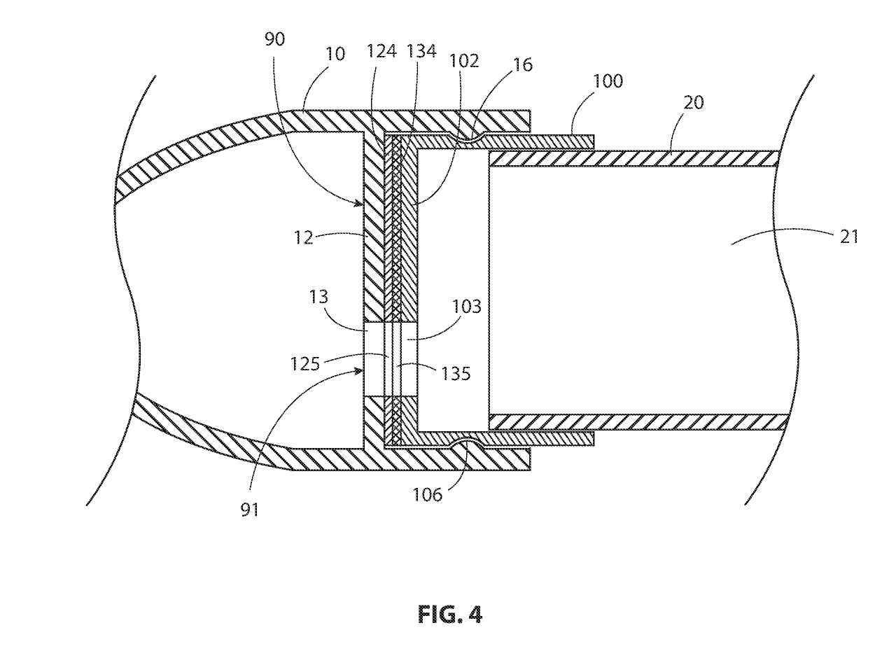

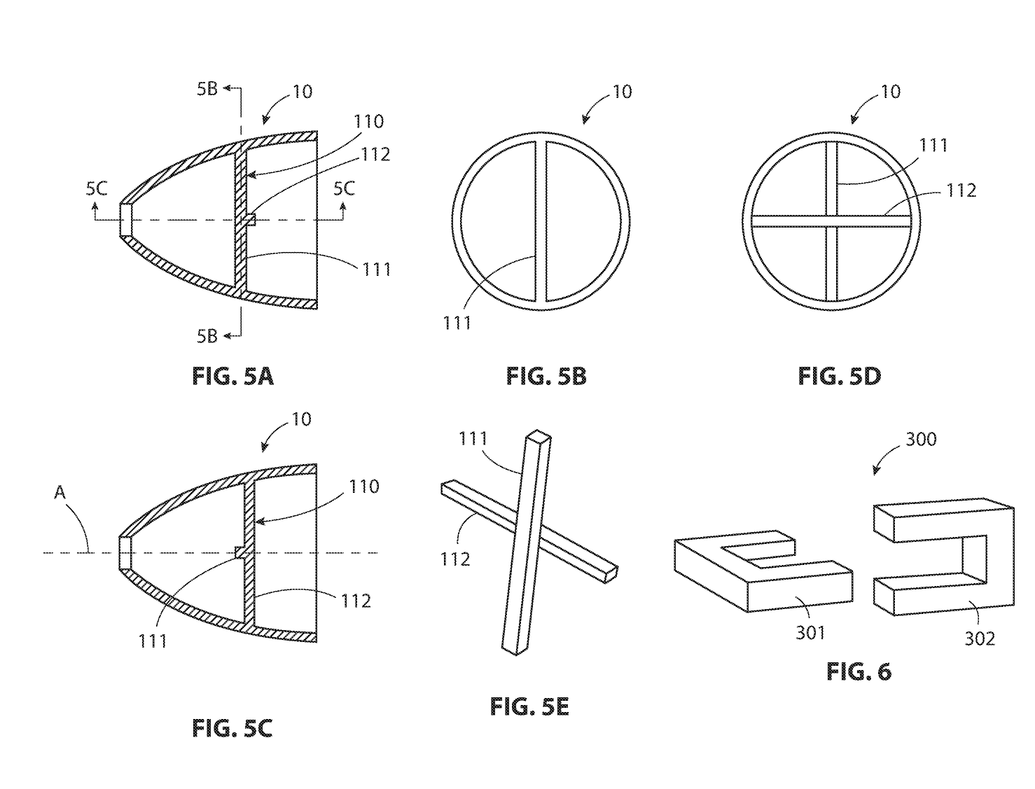

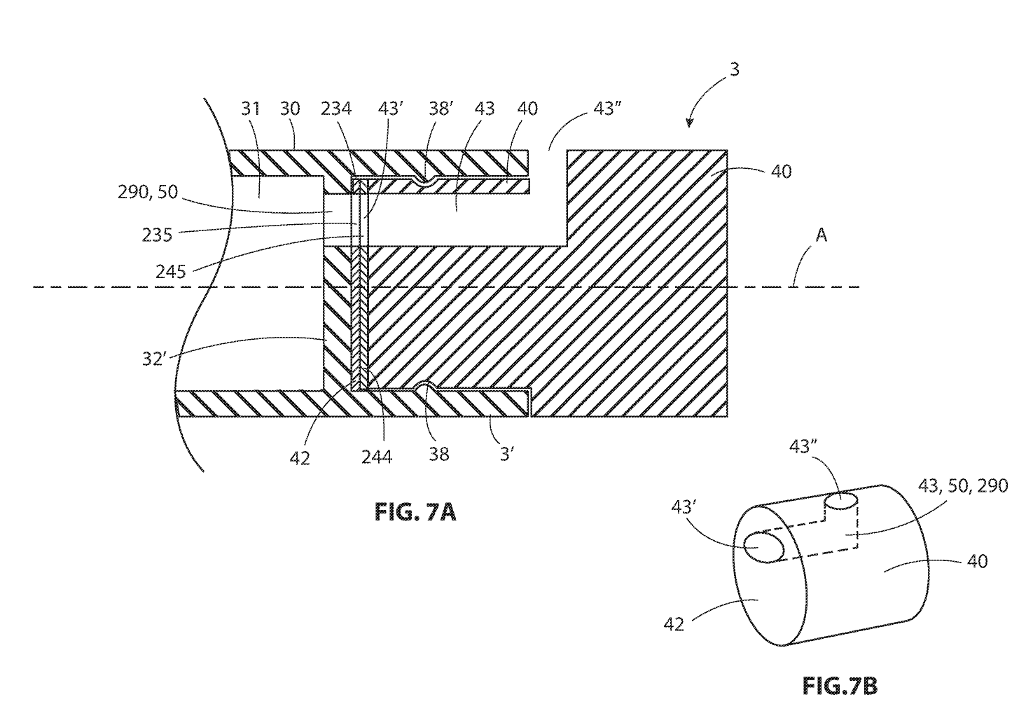

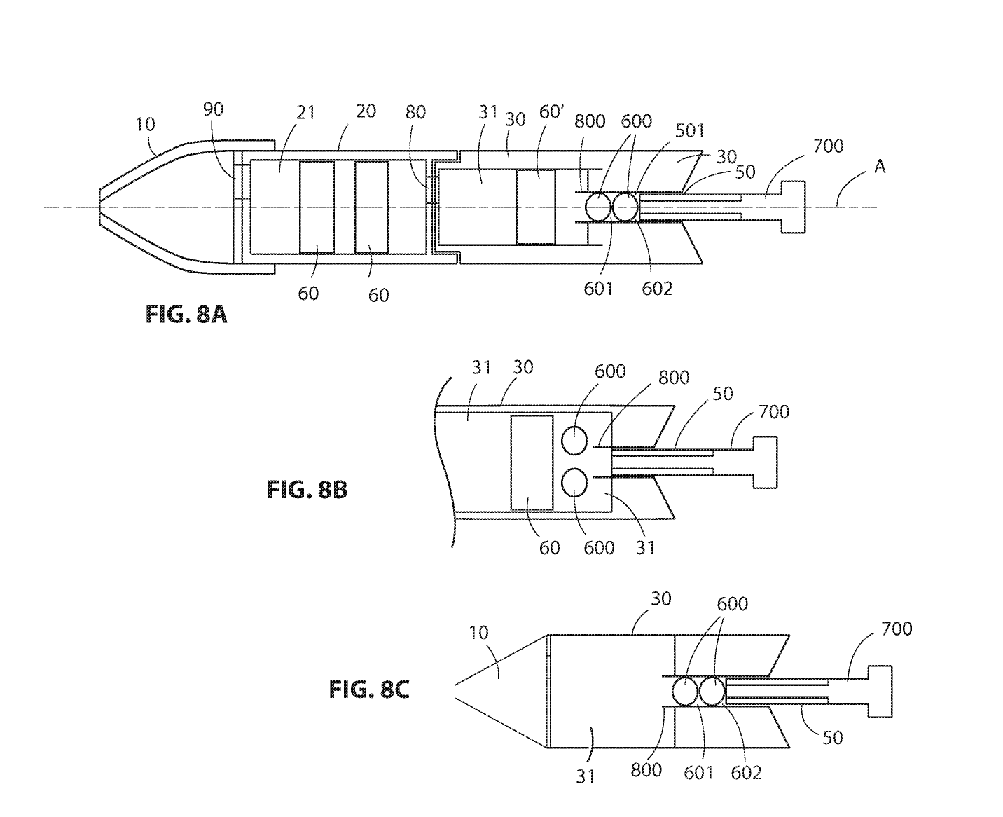

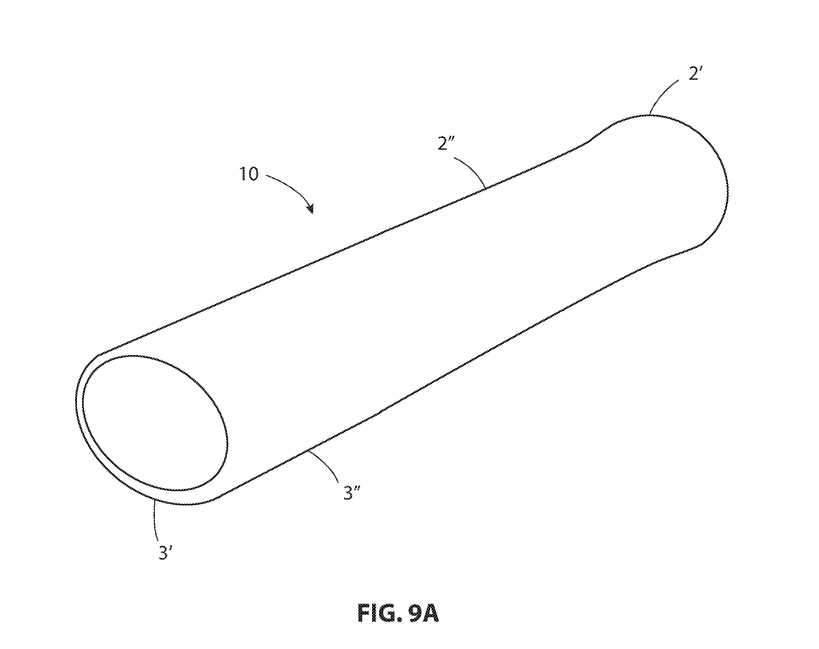

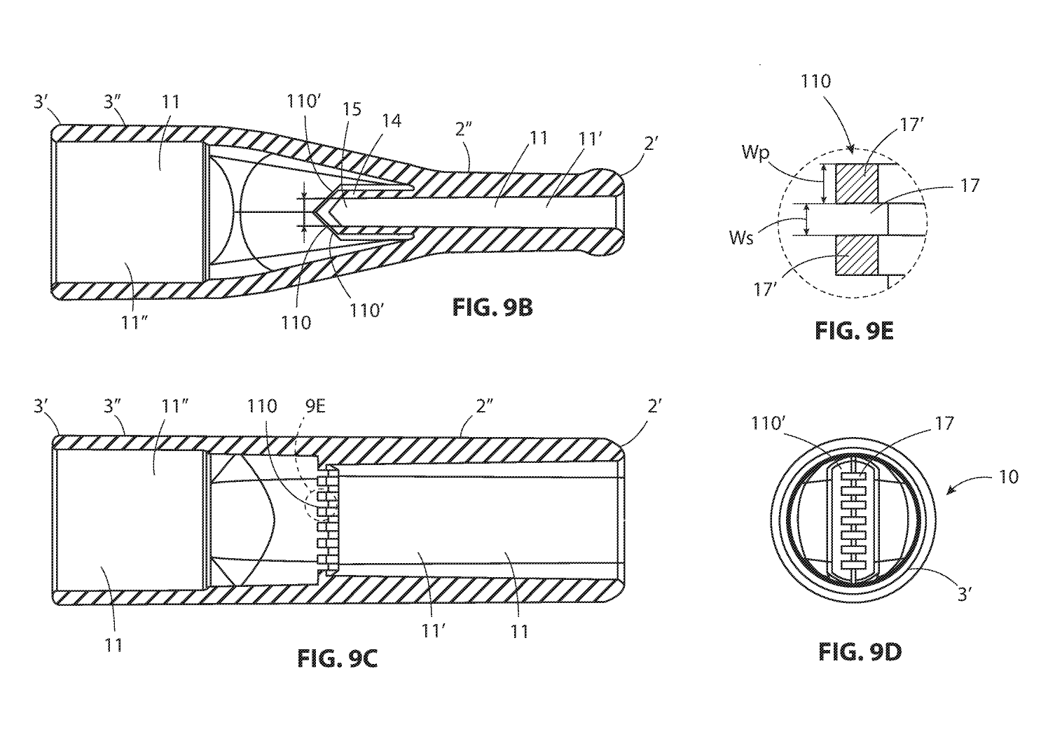

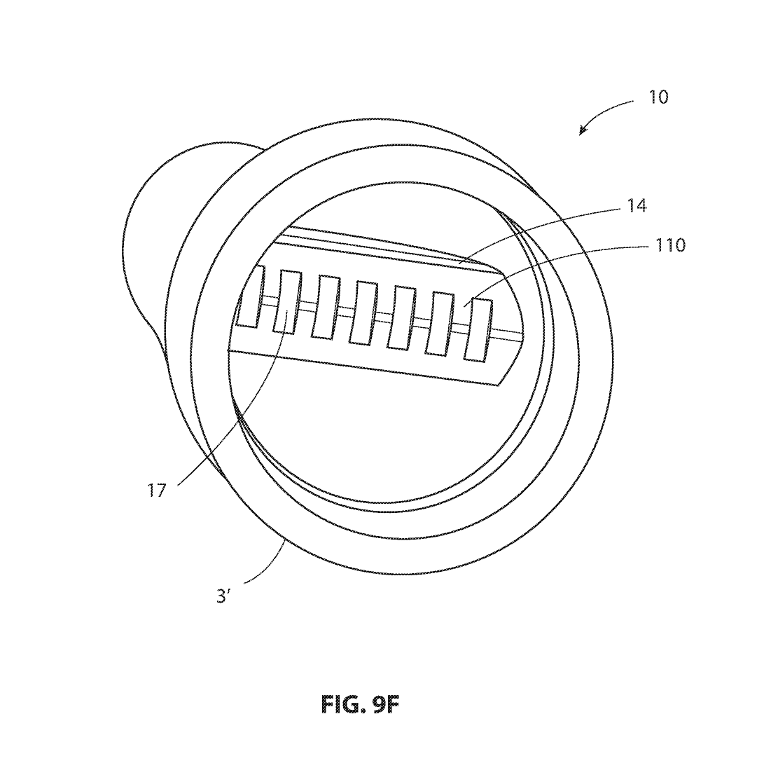

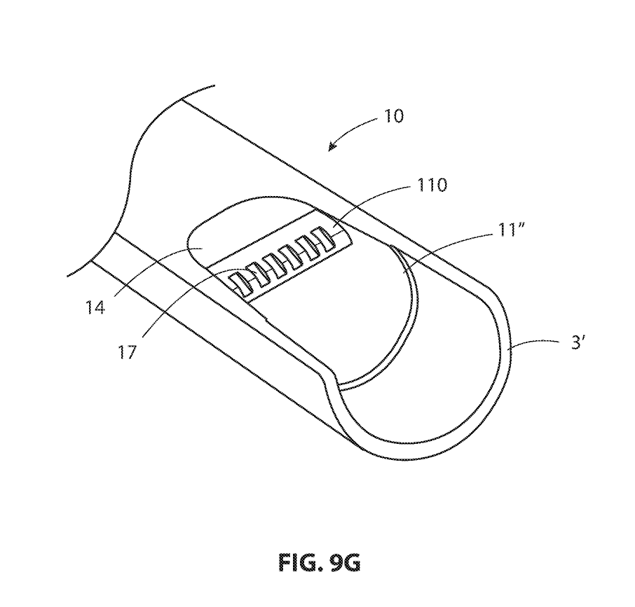

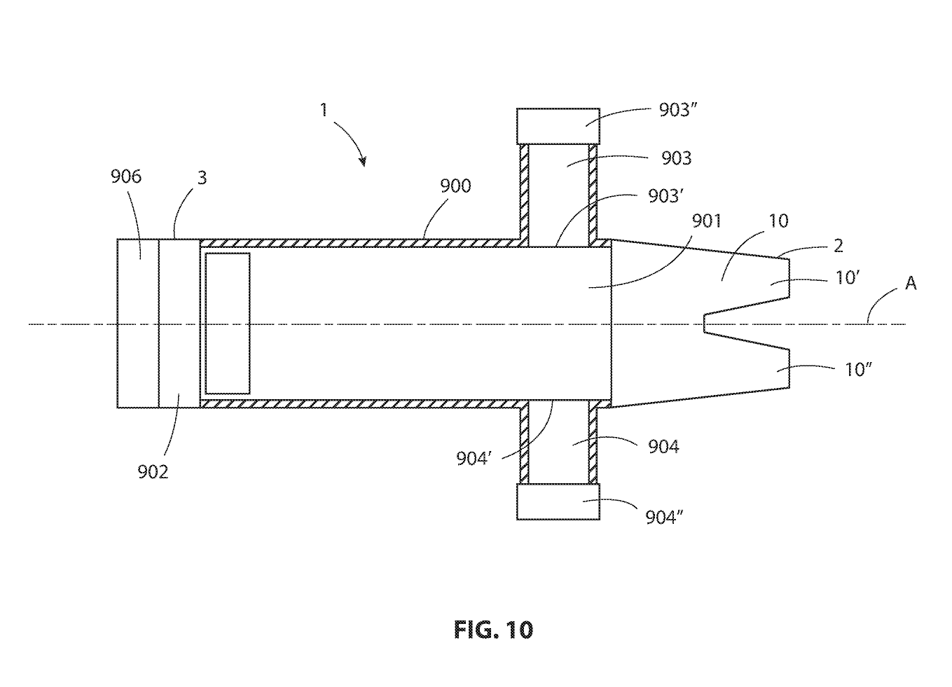

Before use or sometimes also between two different instances of use (multiple use with breaks in between) the first and second compounds must be stored separately, to avoid interaction, which may over time influence the quality of the active and/or the transport compounds. Therefore, the first and second compartments may be separated by a valve 80. The valve 80 provides a seal between the first and second compartments 21, 32 in the closed configuration of the valve 80. In this embodiment, in order to use the inhalator 1, the valve 80 must be opened to provide a passage 81 between the first compartment 21 and the second compartment 31, to activate the inhalator 1. In FIG. 2, the passage 81 is not visible, i.e. the valve 80 is closed or sealed. The opening and closing of the passage 81 is described in relation to FIGS. 3A-C below.

FIG. 3A shows an embodiment of a valve 80 between the first compartment 21 and the second compartment 31. The valve 80 comprises an internal wall 22 formed in the first tubular part 20, and an internal wall 32 formed in the second tubular part 30. The walls 22, 32 are formed transverse to the longitudinal axis A of the first and second tubular parts 20, 30. Each wall 22. 32 comprises a through going opening 23, 33. The openings 23, 33 are located in the respective walls 22, 32 such that when the first tubular part 20 is rotated relative to the second tubular part 30, a passage 81 opens up when the respective openings 23, 33 are aligned rotationally, and such that when the respective openings 23, 33 are not rotationally aligned, the valve 80 is closed, i.e. there is no passage 81. This is illustrated in the exploded view of the walls 22, 32 in FIGS. 3B and 3C.

In FIG. 38, the openings 23, 33 are not aligned and the valve 80 is thus closed. It is clear that if the walls 22, 32 are arranged close together the two walls will provide a barrier for passage when the openings 23, 33 are aligned as shown in FIG. 38.

In FIG. 3C, the openings 23, 33 are aligned and the valve 80 is thus open. It is clear that even when the walls 22, 32 are arranged close together the two openings 23, 33 will provide a passage 81 through the combined walls 22, 32.

The walls 22, 32 of the valve 80 may preferably be provided with a sealing 24, 34, either on one of the walls 22, 32 or both. The sealing 24, 34 is fixedly mounted on the respective walls 22, 32 and each sealing 24, 34 is provided with an opening 25, 35, which opening 25, 35 is permanently aligned with the opening 23, 33 on the respective wall 22, 32. The sealings 24, 34 are preferably formed in a material that is softer than the rigid wall 22, 32 material e.g. a soft polypropylene or rubber or another suitable sealing material, for providing a gas tight, such as an air tight, seal. The sealings 24, 34 are preferably compressed against each other by assembly of the first and second tubular parts 20, 30.

The first and second tubular parts 20, 30 may be connected by a portion of one of the tubular parts 20, 30 being adapted to fit over a potion of the other of the tubular parts 20, 30, and being provided with a mechanism for allowing rotation of the first tubular part 20 relative to the second tubular part 30. Such a mechanism may in one embodiment and as shown be provided by an annular/circumferential lip 26 on an inner surface of the first tubular part 20 cooperating with an annular circumferential groove 36 on an outer surface of the second tubular part 30, see FIG. 3D, showing a detailed view of the potion indicated by circle 8 in FIG. 3A. In other embodiments (not shown) the lip 26 and groove 36 may be reversed between the two tubular parts 20, 30. In yet other embodiments (not shown), the first and second tubular parts 20, 30 may be rotationally connected by a groove being provided on either parts 20, 30, an annular member, such as an O-ring being provided in the cooperating grooves. In yet other embodiments, the above described rotational connections may be combined the O-ring providing an additional air tight sealing.

Stops (not shown) may be provided to limit the rotation between the two parts, e.g. such that at one stop the openings 23, 33, 25, 35 are aligned, i.e. the valve 80 is fully open and at the other stop the openings 23, 33, 25, 35 are not aligned, i.e. the valve 80 is fully closed. Alternatively or additionally, the outer surface of the tubular parts 20, 30 may be provided with markings indicating when the valve is open and closed. In yet an alternative embodiment (not shown) one or both of the tubular parts 20, 30 or portions thereof could be formed in a transparent material. This would allow visual inspection of when the valve 80 is open or closed.

In yet an alternative embodiment one or both of the walls 22, 32 of the valve mechanism 80 may be provided on a cap part that is e.g. press fit on or into the first and/or second tubular parts 20, 30. In this case, the rotation mechanism described above may be located between the cap and the respective tubular part 20, 30. In the case that a cap is arranged on both tubular parts, a rotation mechanism as described above may be located between the caps.

With reference to FIGS. 2 and 4, the connection between the first tubular part 20 and the user interface part 10 is now described. In order to provide an air tight but openable and re-closable seal to preserve the active compound in the first compartment 21, a valve mechanism 90 may further be provided between the first tubular part 20 and the user interface part 10. The valve mechanism may be arranged as described above for the valve mechanism 80 between the first and second tubular parts 20, 30 (and thereby the first and second compartments 21, 31). This is shown in FIG. 2, where the valve 90 is shown in its closed position. An embodiment where a cap 100 is provided between the first tubular part 20 and the user interface part 10 is shown in FIG. 4 in a side sectional view. The cap is preferably press fit on the first tubular part 20 such that it is locked against rotation relative to the first tubular part 20.

The valve 90 between the first compartment 21 and the cap 100 (and thereby the user interface part 10) comprises an internal wall 12 formed in the user interface part 10, and an internal wall 102 formed in the cap 100. The walls 12, 102 are formed transverse to the longitudinal axis A of the inhalator 1. Each wall 12, 102 comprises an opening 13, 103. The openings 13, 103 are located in the respective walls 12, 102 such that, when the user interface part 10 is rotated relative to the cap 100 (and thereby the first tubular part 20), a passage 91 opens up when the respective openings 13, 103 are aligned rotationally, and such that when the respective openings 13, 103 are not rotationally aligned, the valve 90 is closed, i.e. there is no passage 91.

The walls 12, 102 of the valve 90 may preferably be provided with a sealing 124, 134, either on one of the walls or both. The sealings 124, 134 are fixedly mounted on the respective walls 12, 102 and each provided with an opening 125, 135, which opening 125, 135 is permanently aligned with the opening 13, 103 on the respective wall 12, 102. The sealings 124, 134 are preferably formed in a material that is softer than the rigid wall 12, 102 material, e.g. a soft polypropylene or rubber or another suitable sealing material, for providing a gas tight, such as an air tight seal. The sealings 124, 134 are preferably compressed against each other by assembly of the first tubular part 20 and the cap 100.

The user interface part 10 and the cap 100 may be connected by a portion of one of the user interface part 10 and the cap 100 being adapted to fit over a potion of the other of the user interface part 10 and the cap 100, and being provided with a mechanism for allowing rotation of the first tubular part 10 relative to the cap 100. Such a mechanism may in one embodiment and as shown be provided by an annular/circumferential lip 16 on an inner surface of the user interface part 10 cooperating with an annular circumferential groove 106 on an outer surface of the cap 100. In other embodiments (not shown) the lip 16 and groove 106 may be reversed between the user interface part 10 and the cap 100. In yet other embodiments (not shown), the user interface part 10 and the cap 100 may be rotationally connected by a groove being provided on each of the user interface part 10 and the cap 100, an annular member, such as an O-ring, being provided in the cooperating grooves. In yet other embodiments, the above described rotational connections may be combined with the O-ring providing an additional air tight sealing.

Stops (not shown) may be provided to limit the rotation between the user interface part 10 and the cap 100, e.g. such that at one stop the openings 13, 103, 125, 135 are aligned, i.e. the valve 90 is fully open, and at the other stop the openings 13, 103, 125, 135 are not aligned, i.e. the valve 90 is fully closed. Alternatively or additionally, the outer surfaces of the user interface part 10 and the cap 100 may be provided with markings indicating when the valve 90 is open and closed. In yet an alternative embodiment (not shown) one or both of the user interface part 10 and the cap 100 or portions thereof could be formed in a transparent material. This would allow visual inspection of when the valve 90 is open or closed.

In other embodiments (not shown) the valve mechanism 90 may be provided between a cap 100 and the first tubular part 20. The rotation mechanism may be provided between the cap and the first tubular part 20 and the cap 100 may thus be prevented from rotation relative to the user interface part 10, e.g. by a press fit connection.

The solution with the cap 100 corresponds to the one described above for the valve mechanism 80 and the rotation mechanism between the first and second tubular parts 20, 30. Thus it will be appreciated that FIG. 4 may serve to illustrate the connection with a cap described above for the valve mechanism 80 and the rotation mechanism between the first and second tubular parts 20, 30 as well.

In order to provide a flow of active compound from the first compartment 21, the valve 90 must be in an open position. It will be understood that by re-closing valves 80 and 90 after an inhalation, the first compartment 21 is sealed off, whereby the active compound comprised therein may obtain a prolonged preservation.

The cap 100 allows access to the interior of the first part 20 during the manufacture of the inhalator, such that one or more plugs 60 and filings 70 may be placed in the first compartment 21.

A filter (not shown) may be provided inside the user interface part 10 to prevent dust, rice grains or other components from e.g. the filling 70 to enter the user's mouth or nose. However, instead of a filter a simple lattice 110 comprising to crossed beams 111, 112 may suffice. The lattice does not reduce the airflow or the taste experience (it doesn't block the aroma compounds) as does a filter, but prevents larger materials from being drawn into the mouth or nose of the user. FIGS. 5A-E illustrates such a lattice 110, where the two beams are formed perpendicular to each other and to the longitudinal axis A of the inhalator 1. The beams may be formed with a gap between them (in the longitudinal direction A), but in a preferred embodiment, the two beams are formed as single unit, i.e. they are connected at their intersection. Although the lattice 110 may be a component that that is assembled into the user interface part 10. In a preferred embodiment the user interface part 10 and the lattice 110 are formed as one integral part in an injection molding process.

A tool 300 comprising two cooperating tool parts 301, 302, suitable for providing a lattice as shown in FIGS. 5A-E is shown in FIG. 6. The tool parts 301, 302 are two identical fork-like structures, that are angled 90 degrees relative to each other, and that may be inserted into the mold (not shown) for forming the tubular user interface part 10 to form the lattice shown in FIGS. 5A-E.

In order to preserve the transport compound in the second compartment until use and possibly in between instances of use, the second compartment is provided with a valve mechanism 200 at the distal end 3′ of the second tubular part 30. The valve mechanism 200 is shown in FIG. 7 and provides an openable and re-closable passage 290 that may be an embodiment of the ventilation ports 50 discussed above and shown in FIG. 2. When the valve 200 is opened (and valves 80 and 90 are also open), it is possible to suck ambient air through the inhalator 1 by providing suction at the user interface part (or by blowing air in via the ventilation port(s) 50). When the valve 200 is closed the second compartment is sealed off from the ambient air (provided that one or both the valves 80, 90 are also closed), and in any event it will no longer be possible to have a flow of air/gas through the inhalator 1.

The valve mechanism 200 may be arranged as described above for the valve mechanisms 80, 90. The valve 200 is preferably provided between the second compartment 31 and the activator part 40. The valve 200 comprises an internal wall 32′ formed in the distal end of the second tubular 30, and an wall 42 formed on the activator part 40. The walls 32′. 42 are formed transverse to the longitudinal axis A of the inhalator 1. The wall 42 on the activator part 40 is preferably and proximally facing end wall of the activator part 40, The activator part 40 may be solid or may be hollow. The wall 32′ on the second tubular part 30 comprises an opening 33′. The activator part 40 further has a channel 43 that opens into the end wall 42 of the activator part 40 and into a side wall 40′ of the activator past 40 such that the channel 43 connects an opening 43′ in the end wall 42 of the activator part 40 and an opening 43″ in the side wall 40′.

The openings 33′. 43′ are located in the respective walls 32′, 42 such that, when the second tubular part 10 is rotated relative to activation part 40, a passage 290 opens up when the respective openings 33′, 43′ are aligned rotationally, and such that when the respective openings 33′, 43′ are not rotationally aligned, the valve 200 is closed, i.e. there is no passage 290.

The walls 32′, 42 of the valve 200 may preferably be provided with a sealing 234, 244, either on one of the walls or both. The sealings 234, 244 are fixedly mounted on the respective walls 32′, 42 and each provided with an opening 235, 245, which opening 235, 245 is permanently aligned with the opening 33′, 43′ on the respective wall 32′, 42. The sealings 234, 244 are preferably formed in a material that is softer than the rigid wall 32′, 42 material, e.g. a soft polypropylene or rubber or another suitable sealing material, for providing a gas tight, such as an air tight seal. The sealings 234, 244 are preferably compressed against each other by assembly of the second tubular part 30 and the activator part 40.

The second tubular part 30 and the activation part 400 is further connected by a portion of the second tubular part 30 is adapted to fit over a portion of the activation part 40, and by being provided with a mechanism for allowing rotation of the second tubular part 30 relative to the activation part 40. Such a mechanism may in one embodiment and as shown in FIG. 7A be provided by an annular/circumferential lip 36′ on an inner surface of the second tubular part 30 cooperating with an annular circumferential groove 46 on an outer surface of the activation pad 40. In other embodiments (not shown) the lip 38′ and groove 46 may be reversed between the second tubular part 30 and the activation part 40.

In yet other embodiments (not shown), the second tubular part 30 and the activation part 40 may be rotationally connected by a groove being provided on each of the second tubular part 30 and the activation part 40, an annular member, such as an O-ring, being provided in the cooperating grooves. In yet other embodiments, the above described rotational connections may be combined with the O-ring providing an additional air tight sealing.

Stops (not shown) may be provided to limit the rotation between the second tubular part 30 and the activation part 40, e.g. such that at one stop the openings 33′, 43′, 235, 245 are aligned, i.e. the valve 200 is fully open, and at the other stop the openings 33′, 43′, 235, 245 are not aligned, i.e. the valve 200 is fully closed. Alternatively or additionally, the outer surfaces of the second tubular part 30 and the activation part 40 may be provided with markings indicating when the valve 200 is open and closed. In yet an alternative embodiment (not shown) one or both of the second tubular part 30 and the activation part 40 or portions thereof could be formed in a transparent material. This would allow visual inspection of when the valve 200 is open or closed.

In other embodiments (not shown) the valve mechanism 200 may be provided between a cap on the second tubular part 30 and the activation part 40. The rotation mechanism may in this case be provided between the cap and the second tubular part 30, in which case the activation part and the cap would have to be locked against rotation relative to each other, e.g. via a press fit. Alternatively, the rotation mechanism may could be provided between the cap and activation part. In this case the cap would be locked against rotation relative to the second tubular part 30, e.g. by a press fit.

In order to provide a flow through the inhalator the valve 200 must be in an open position. It will be understood that by re-closing valves 200 and 80 after an inhalation, the second compartment 31 is sealed off, whereby the transport compound comprised therein may obtain a prolonged preservation.

An alternative valve mechanism may be provided the distal end of the inhalator. For example the activation part 40 may be secured in the second tubular part via cooperating threading on the two parts, and where a turning of the activation part relative to the second tubular part will cause a displacement in the longitudinal direction along axis A, that may open up a channel, e.g. by aligning openings. In yet an alternative embodiment, the activation part 40 may be translational movable relative to the second tubular part 30, pushing the two parts away from each other to open a vent port 50.

In embodiments (not shown) the activation part 40 or portions thereof may be formed in a diffusion open material. When the activation portion 40 or portions thereof is provided in a diffusion open material there may be provided a seat (not shown) between the ambient air and at least the second compartment. The seal may e.g. be provided in the second compartment. The seal provides an airtight barrier to preserve the compounds provided in the second compartment until the seal is broken/opened by moving the activation part 40. Alternatively, the diffusion open material activation part 40 may at its proximal end be provided with a seal that is not diffusion open, e.g. in the form of a coating or a plate attached to the end face. The seal in this form may seal the second compartment by cooperation with parts on the inner wall and open by be moved relative to these parts.

The allignable openings described above may preferably be oval in shape, but may take other shapes as well.

In an embodiment (not shown) the activation part 40 may be provided with a pen function allowing a user to use the inhalator as a pen. Also, the activation part 40 may be equipped with a battery and a light source e.g. a diode.

Especially for certain medical uses of the inhalator 1, the central connection, i.e. valve 80 and the connection between the between the first and second tubular parts 20, 30 may be replaced by a fixed barrier formed by a diffusion open material, e.g. molecular sieves, active coal or a filter or a polyethylene plug (cylindrical or spherical), as described above (however without ‘loading’ any compounds there into). Such a barrier may also be formed by small polyethylene spheres 600 filling out a segment of a chamber, e.g. a segment 501 of the above mentioned second compartment 31. Such barrier-variations may especially be applied when the transport compound in the second compartment is not so volatile or eager to react with the compound in the first compartment Thus, the barrier type and characteristics may be chosen dependent on the reactivity of the active and transporter compounds.

In such embodiments the first and second tubular parts may not be rotatable in respect of each other. Actually, the first and second tubular parts may then be formed as a single tubular item, the above mentioned barrier separating the first and second compartments 21, 31. The active compound is still placed in the proximal first compartment 21, and the transport compound is still placed in the distal second compartment 31 so that a gas (air) is drawn through the chamber with the transport compound first, drawing these through the barrier and into and through the first compartment 21 with the active compound, and into the user via the user interface part 10.

The inhalator may for example be used to transport (e.g. nanotechnologically) coated medical compounds orally or nasally into a users body. Example: A coated molecule can be entered in to the body to a targeted location when the e.g. the coating is removed by the body, e.g. by the immune system of the body to free the medical compound from the coating at the right time and location. For example a medically active compound can be transported to the brain where the coating is gradually dissolved or reduced during the transport or upon arrival. It the same medically active compound had not been coated, it would have been dissolved or otherwise attacked.

With a closed compartment containing the medicine, a coater, or a coated medicine or a transporter for dilution, and with two side compartments for injection two other coaters or medicines, a multiple compound solution may be formed, with a mixture of stable compounds, that after the mixture will become unstable/active, where after a front loader may be activated with spheres (see below) to deliver the transport compound (e.g. with eucalyptus mixed with methanol/alcohol) into a polypropylene plug, as shown above.

In a further aspect the invention also relates to an inhalator as shown in FIGS. 8A-8, in which the second compartment 31 communicates with a third compartment 501. Most parts of this inhalator 1 are similar to the ones described in connection with the embodiments describes above in connection with FIGS. 1-7, and like components have the same reference numbers.

The third compartment 501, in an un-activated state of the inhalator contains two or more spheres 600 the spheres fill out the third compartment 501 and seats of chambers 601, 802 between the spheres and the inner wall of the compartment 501 and—in the case of chamber 602, a pin 700. The spheres 600 are formed in a material that does not allow diffusion of gas or liquids. Thereby, the chambers 601, 602 may contain fluid solutions, e.g. an active compound in one chamber 601 and a transporter in the other chamber 602. By pushing the spheres 600 into the second compartment 30, using a pin 700 (and resulting in the situation shown in FIG. 8, the fluid in the chambers 601, 602 will mix). Thus, the second compartment 31 will become a mixing chamber. The pin 700 corresponds to the activation part 40, described above.

In order to prevent a backflow of fluid from the second compartment 31, the transition between the second compartment and the third compartment 501 is provided with an, annular flange 800 extending in a direction parallel to the longitudinal axis A.

The annular flange 800 may also be applied in an inhalator as in FIG. 8A-B but where there is no first compartment 20, and basically as in FIG. 8C in all of the versions described in connection with FIG. 8 the one or more venting channel 50 is provided at the distal side, e.g. by utilizing the principles of the valve 20 shown in FIG. 200.

The pin 700 shown in FIG. 8A-C may be used, as described above, to push said spheres 600 into the second chamber for mixing of compounds contained in the chambers formed by the spaces between the spheres and the inner wall of the third compartment. But, in other embodiments (not shown) it may also be used for pushing or penetrating a seal, e.g. in the form of a shim, from a closed state to an open state. The pin 700 may in either case be pushed into the inhalator 1 or it may be connected to the inhalator by a threading (not shown).

The pin 700 or at least a proximal portion thereof, may be formed in a diffusion open material, e.g. polypropylene, an may thus have the additional function as a filtered air entrance port. Further it may thus collect possible fluid extracts from the inhalator and thus prevent leakage of fluids there from. A diffusion open material pin 700 may further distribute such fluid over a larger surface area, which will increase the evaporation of the fluid.

When the pin 700 or portions thereof is provided in a diffusion open material there may be provided a seal (not shown) between the ambient air and at least the second compartment. The seal may e.g. be provided in the third compartment, in the transition between the second and the third compartment, or it may be provided in the second compartment. The seal provides an airtight barrier to preserve the compounds provided in the third and/or second compartment 31 until the seal is broken/opened by moving the pin 700. Alternatively, the diffusion open material pin 700 may at its proximal end be provided with a seal that is not diffusion open. e.g. in the form of a coating or a plate attached to the end face. The seal in this form may seal the third compartment by cooperation with parts on the inner wall and open by be moved relative to these parts.

FIGS. 9A-E show another embodiment of a user interface part 10 in the form of a mouthpiece. The user interface part 10 has a proximal end 2′ shaped to interact with a user's lips and mouth. The user interface part 10 further has a distal end 3′ adapted for connection to a tubular part 20, 30 of an inhalator as described above. In the embodiment shown the figure the distal 3′ end is cylindrical and adapted for engaging a cylindrical tubular part 20, 30. In other embodiments, a cross-section of the user interface part 10 may be different from circular, provided that it is adapted to correspond to the shape of the tubular part 20, 30 to which it is to be connected. The proximal end 2′ is flattened relative to the distal end 3′ in order to provide an ergonomic contact with a users mouth. A passage 11 is formed through the user interface part. A filter 110 is formed in the passage 11 to prevent particles or dust to pass through the user interface part. The filter 110 is preferably formed on a projection 14 formed inside a distal portion 3″ of the user interface part 10, and in extension of a portion 11′ of the passage 11 that stretches in a proximal portion 2″ of the user interface part 10. The fitter 110 forms a grid that extends across the opening 15 of portion 11′ into a portion 11″ of the passage 11 that stretches in the distal portion 3″ of the user interface part 10. The filter may form an integral part of the projection 14, or it may be formed as a separate part fitted on or in the projection 14. The filter may preferably be formed as slotted V-shaped plate 110′, having slots or openings 17 and intermediary plate portions 17′ interchangingly arranged between each others. Preferably, the width of the slots Ws is the same as the width Wp of the intermediary plate portions 17′. This width may in an exemplary embodiment be 0.4 mm. The V-shape of the filter 110 “points” in the distal direction.

The user interface part 10 may further comprise components for a valve 90 arrangement like the one shown in FIGS. 2 and 4.

FIG. 9F, in a perspective view, shows a view of the distal end 3′ of the user interface part 10. FIG. 9G, in perspective and partly cut out view, show details of the filter arrangement of the user interface part 10.

FIG. 10 shows an embodiment of an inhalator 1 according to an aspect of the invention, which is particularly useful for medical applications in order to bring a medical compound into contact with tissue in e.g. the brain. There is a short distance between the nasal cavity and the brain. Therefore, medical compounds, e.g. anti depressive medication, may easily be brought close to the brain tissue though nasal delivery. Thereby the efficiency of delivery may be considerably be improved. The inhalator 1 in FIG. 10 has a user interface part 10 especially adapted for nasal delivery, by having two delivery outlets 10′, 10″ adapted for interfacing with the nostrils of a user/patient. As described above, the user interface part may be provided with a filter (not shown). In case oral delivery is preferred, a user interface part as described above in connection with the FIG. 1-9 embodiments may be provided.

The user interface part 10 is connected to a tubular device part 900. A first compartment, which could here be called a mixing compartment 901, is provided in the tubular device 900.

A valve mechanism (not shown) may be provided between the user interface part 10 and the mixing compartment 901, in the same way as described for the FIG. 1-9 embodiments above.

The inhalator 1 is constructed such that a gas may be drawn through the mixing compartment 901 as described above, from a distal end 3 of the inhalator 1 to the proximal end 2 by a user/patient, or by providing a flow of gas from a source (not shown).

The mixing compartment 901 may be empty or it may be loaded with e.g. medical compound, a coater, or a coated medicine or a transporter for dilution of the medical compound. One or two side compartments 903, 904 are formed in a lateral direction relative to a longitudinal axis A of the inhalator. The one or two side compartments 903, 004 may contain a medical compound, a coater, or a coated medicine. The one or two side compartments 903, 904 are isolated from the mixing compartment 901 until use is desired, e.g. by valve mechanism as described above or by a dissolvable or breakable barriers 903′. 904′. When an delivery is to be made the compound(s) contained in the one or two side compartments 903, 904 may be forced into the mixing compartment 901 by suitable activation mechanisms 903″, 904″, e.g. as described in connection with the FIG. 1-9 embodiments above. Thereby a multiple compound solution may be formed, e.g. with a mixture of stable compounds, that after the mixture will become unstable/active. The mixture may then be transported into the user/patient in the manner described above

Preferably, a second compartment, here called a transport compound compartment 902 is connected to the mixing compartment 901 at the opposite end (with respect to the user interface part 10), distal end 3. The transport compound compartment 902 contains a transport compound as 10, described above. The transport compound compartment 902 may be connected to the mixing compartment in any of the ways described in connection with the FIG. 1-9 embodiments above. The transport compound (e.g. with eucalyptus mixed with methanol/alcohol) may be contained in a polypropylene plug, as described above.

In one embodiment two side compartments 903, 904 and one transport compound compartment 902 are provided in connection with the mixing compartment. One side compartment contains a coater and the other contains a medical compound.

The Inhalator 1 may for example be used to transport (e.g. nanotechnologically) coated medical compounds orally or nasally into a users body. A coated molecule can be entered in to the body to a targeted location. The coating is gradually dissolved or reduced, e.g. by the immune system, during the transport through the body, or upon arrival to the target site, it the same medically active compound had not been coated, it would have been dissolved or otherwise attacked. For example a medically active compound can be transported to the brain in this manner.

Although the teaching of this application has been described in detail for purpose of illustration, it is understood that such detail is solely for that purpose, and variations can be made therein by those skilled in the art without departing from the scope of the teaching of this application.

The term “comprising” as used in the claims does not exclude other elements or steps. The term “a” or “an” as used in the claims does not exclude a plurality. The single processor or other unit may fulfill the functions of several means recited in the claims.

1. An inhalator having a proximal end being closest to a user during use of the inhalator and having a distal end being furthest away from the user during use of the inhalator, the inhalator comprising
a first compartment containing an active compound;
a second compartment containing a transporter; and
an activation part configured for regulating an air flow through the first and the second compartment of the inhalator, the activation part being arranged at a distal end of the second compartment,
wherein the first compartment is arranged between a user interface part and the second compartment such that when a user sucks air or a gas is forced through the first and the second compartment of the inhalator, the transporter is drawn from the second compartment and into and through the first compartment, whereby at least a part of the active compound is captured by or mixed with the transporter in the first compartment and flows into the user interface part and the user.

2. The inhalator according to claim 1,
wherein a first valve is arranged between the first and second compartments.

3. The inhalator according to claim 1, wherein
a second valve is arranged between the first compartment and the user interface part.

4. The inhalator according to claim 1,
wherein a third valve is arranged between the second compartment and the activation part.

5-7. (canceled)

8. The inhalator according to claim 2, wherein a second valve is arranged between the first compartment and the user interface part.

9. The inhalator according to claim 2, wherein a third valve is arranged between the second compartment and the activation part.
